(19) United States
(12) Patent Application Publication
(10) Pub. No.: US 2007/0072894 A1
(43) Pub. Date: Mar. 29, 2007

(54) Synthesis of pyrroloquinoline quinone (PQQ)

(76) Inventors: J. Vern Kempf, Concord, OH (US); Damodara Gopal, Concord, OH (US); Walter Stalzer, Concord, OH (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/387,014

(22) Filed: Mar. 21, 2006

Related U.S. Application Data
(60) Provisional application No. 60/664,989, filed on Mar. 24, 2005.

(51) Int. Cl. A61K 31/4745; C07D 471/02

(52) U.S. Cl. 514/292; 546/82

(57) ABSTRACT

The invention relates to a novel nine step process for synthesizing PQQ (methoxatin). This process is efficient and reliably provides PQQ in excellent purity and high yield.

### RELATED APPLICATIONS

This application claims priority to U.S. Ser. No. 60/664,989 filed Mar. 24, 2005 which is incorporated herein by reference in its entirety.

### FIELD OF THE INVENTION

The present invention is directed to a process which utilizes a triacid potassium salt for the synthesis of 2,7,9-tricarboxy-Pyrrolo Quinoline Quinone (“PQQ”), a redox cofactor that provides a means for treating various diseases.

### BACKGROUND OF THE INVENTION

PQQ, also termed methoxatin (or 2,7,9,-tricarboxy-1H-pyrrolo(2,3-f)quinoline-4,5-dione or 4,5-dioxo-4,5-dihydro-1H-pyrrolo[2,3-f]quinoline-2,7,9-tricarboxylic acid), was isolated in 1979 from methylotrophic bacteria. (Salsibury et al., 1979, Nature 280:843-844). PQQ (1) has the following formula and can be reversibly reduced to the semi-quinone or fully reduced hydroquinone, PQQH₂ (2):

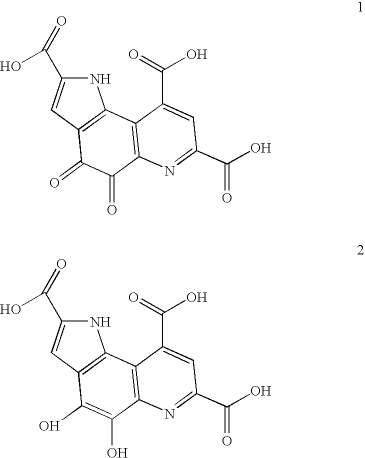

PQQ (1) functions as a redox factor for a number of bacterial enzymes, has vitamin properties in mammals, and is known to inhibit aldose reductase and reverse transcriptases (including HIV-1). (Martin et al., Helv. Chem. Acta 76 (1993) 1667). More specifically, PQQ has been implicated in processes such as: 1) antioxidant protection against glutocorticoid-induced cataract accompanied by maintenance of reduced glutathione levels (Nishigori et al., 1989, Life Sci. 45:593-598); 2) protection against hepatotoxin-induced liver injury (Watanabe et al., 1988, Curr. Therap. Res. 44:896-901; Urakami et al., U.S. Pat. No. 5,061,711); 3) Pharmacology 37:264-267); 4) anti-inflammatory action against carrageenin-induced rat paw edema (Hamagishi et al., 1990, J. Pharmacol. Exp. Therap. 255:980-985); 5) control of NMDA receptor-mediated neuronal injury (Aizenman et al., U.S. Pat. No. 5,091,391); and 6) inhibition of osteoclast cell formation and bone resorption (Hauschka et al., U.S. Pat. No. 5,616,576). There is also speculation that PQQ (1) may be useful in treating diseases such as: inflammatory joint disease, hemolytic anemia, neuromotor defects, disease of the liver, and osteoporosis (Gallop et al., U.S. Pat. No. 5,460,819).

As a natural substance, PQQ (1) can be prepared from biological production (Urakami et al., U.S. Pat. No. 5,061,711; Narutomi et al., U.S. Pat. No. 4,898,870; and Ameyama et al., U.S. Pat. No. 4,994,382). However, like other natural substances, PQQ (1) can be both difficult and expensive to obtain by biological production and isolation. Alternatively, PQQ (1) has been chemically synthesized as shown in Scheme 1 (Corey et al., J. Am. Chem. Soc. 103 (1981) 5599; Martin et al., Helv. Chem. Acta 76 (1993) 1667). 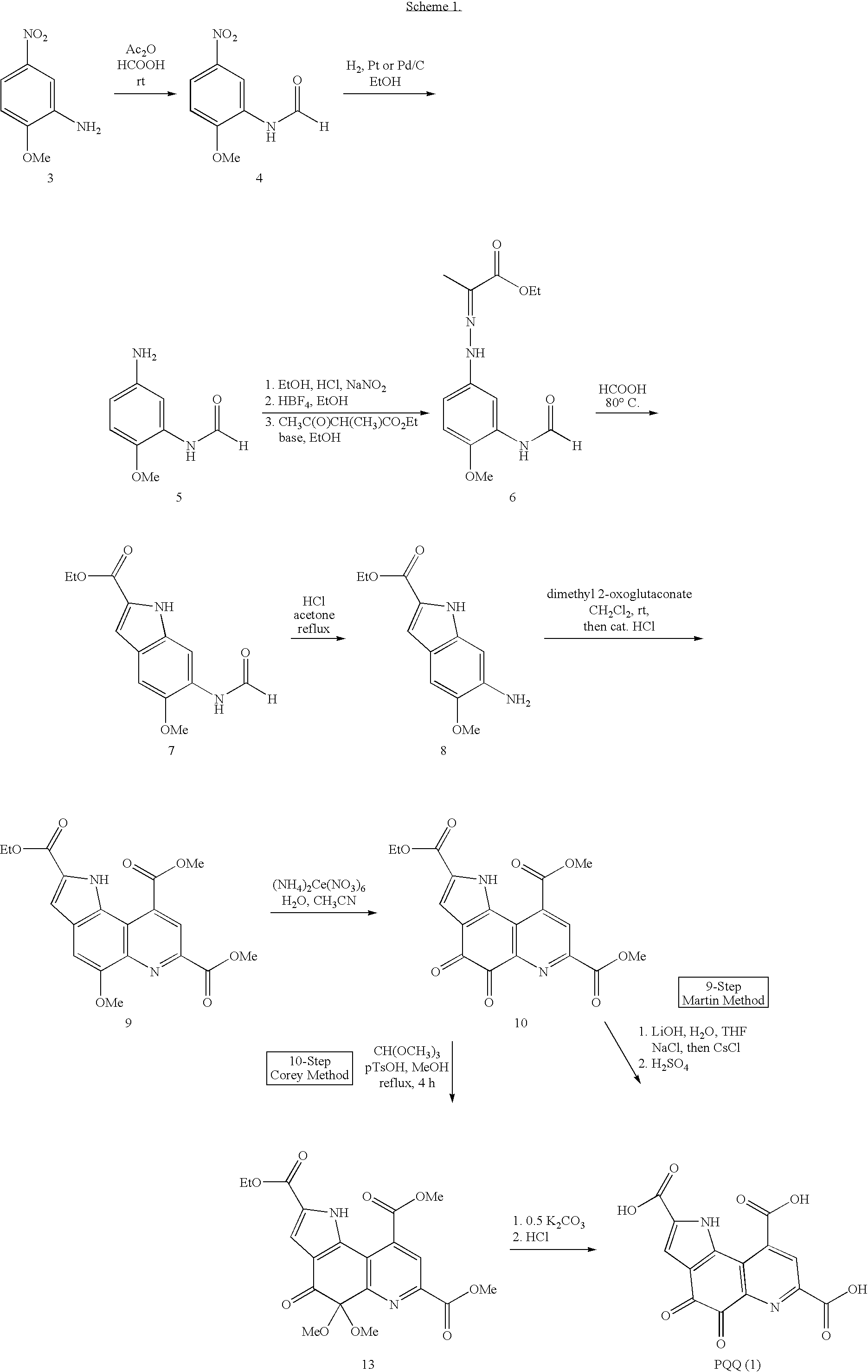
Corey and Tramontano first prepared PQQ (1) on a 50 milligram scale using a ten step synthesis (Corey et al., J. Am. Chem. Soc. 103 (1981) 5599). Martin later prepared 1 using a route similar to that of Corey, but adapted the synthesis to be a larger, semi-pilot plant scale method. Martin modified the reactions in the last steps of the synthesis and was able to reduce the total number of synthetic steps from ten to nine. (Martin et al., Helv. Chem. Acta 76 (1993) 1667). Other synthetic methods have also been reported for the preparation of 1. (See, Freeman et al., WO 94/01142).

The methods of Martin and Corey are deficient at the preparation of PQQ (1) on a multi-gram scale. The method of Corey prepares PQQ (1) on only on a small scale (50 milligrams) and requires an additional synthetic step relative to Martin. And, although Martin's method can be used to prepare PQQ on a multi-gram scale, it requires a tedious, two stage isolation procedure in the final step of the synthesis. Accordingly, there is a need for an improved large scale process to synthesize PQQ (1) more efficiently in high yield and excellent purity.

### SUMMARY OF THE INVENTION

The present invention is directed generally to a process for the improved synthesis of PQQ (1). More specifically, the invention is directed to a method for synthesizing PQQ (1) and its intermediates, derivatives, and analogs more reliably and efficiently on a multi-gram scale in high yield and in high purity. The process creates a triacid salt, 11, comprised of the metal potassium as shown in Formula I: 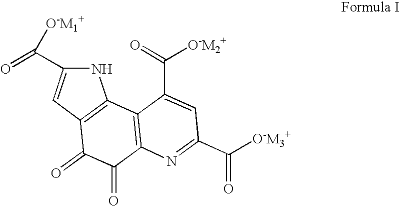
where M₁ is hydrogen or potassium; M₂ is hydrogen or potassium; and M₃ is hydrogen or potassium. In one embodiment, M₁, M₂, and M₃ are not each hydrogen. In a second embodiment, two of M₁, M₂, and M₃ are not hydrogen. In another embodiment, M₁, M₂, and M₃ are each potassium.

The present invention teaches a method for the final step of the synthesis of PQQ involving treating 4,5-dioxo-4,5-dihydro-1H-pyrrolo [2,3-f]quinoline-2,7,9-tricarboxylic acid 2-ethyl ester 7,9-dimethyl ester (10) in tetrahydrofuran with base (e.g., lithium hydroxide) at low temperature, which, upon adding a salt and hydrochloric acid, forms the triacid salt of PQQ comprised of a single metal (e.g. potassium) under controlled pH. Dissolution of the triacid salt in sulfuric acid and addition of the resulting solution to cold water affords PQQ (1).

Unlike the previously reported method of Martin, where PQQ (1) is prepared from 4,5-dioxo-4,5-dihydro-1H-pyrrolo[2,3-f]quinoline-2,7,9-tricarboxylic acid 2-ethyl ester 7,9-dimethyl ester (10), hydrolyzed at room temperature, treated with two separate salts and isolated in two steps as two separate triacid salts (sodium and cesium), the process of the present invention eliminates this cumbersome two step procedure. The present invention uses a simple, reliable method to isolate PQQ as a triacid salt comprised of a single metal in excellent purity and under conditions of carefully controlled temperature and pH.

In one embodiment, the method of PQQ synthesis comprises treating 4,5-dioxo-4,5-dihydro-1H-pyrrolo[2,3-f]quinoline-2,7,9-tricarboxylic acid 2-ethyl ester 7,9-dimethyl ester in tetrahydrofuran with base at low temperature, adding a salt, followed by adding hydrochloric acid. In a preferred embodiment, the triacid salt is comprised of the single metal potassium. In one embodiment, the triacid salt is formed using lithium hydroxide and a halide salt. In a further embodiment, the triacid salt is formed using the halide salt, potassium chloride. In another embodiment, the triacid is formed using lithium hydroxide and a carbonate salt. In a further embodiment, the triacid salt is formed using the carbonate salt, potassium carbonate. In another embodiment, the triacid is formed using an ammonium salt. In a further embodiment, the triacid is formed using the ammonium salt, ammonium chloride.

In the present invention, the temperature of the reaction mixture is maintained at or below 17° C. In a preferred embodiment, the temperature of the reaction mixture is maintained at 16-17° C. The pH of the reaction mixture is carefully adjusted and maintained at or below 6. In a preferred embodiment, the pH is adjusted and maintained at 5.3. In one embodiment, the addition order of reagents to the reaction flask is: 4,5-dioxo-4,5-dihydro-1H-pyrrolo[2,3-f]quinoline-2,7,9-tricarboxylic acid 2-ethyl ester 7,9-dimethyl ester, tetrahydrofuran, lithium hydroxide, potassium chloride, and hydrochloric acid. In a preferred embodiment, the compound is comprised of the single metal potassium. The triacid salt is converted to PQQ upon dissolving the salt in sulfuric acid and adding the acidic solution to water.

In one embodiment, a method for the synthesis of PQQ comprises the following: step a, treating 2-methoxy-5-nitroaniline with formic acid in the presence of acetic acid and water to form N-(2-methoxy-5-nitrophenyl)formamide; step b, treating N-(2-methoxy-5-nitrophenyl)formamide with hydrogen in the presence of palladium and dimethyl formamide to form N-(5-amino-2-methoxyphenyl)formamide; step c, treating N-(5-amino-2-methoxyphenyl)-formamide with sodium nitrite and fluoroboric acid in the presence of water and ethanol, followed by adding ethyl 2-methylacetoacetate and sodium acetate in the presence of water to form ethyl 2-[(3-formylamino-4-methoxyphenyl)hydrazono]-propionate; step d, treating ethyl 2-[(3-formylamino-4-methoxyphenyl)hydrazono]-propionate with formic acid to form ethyl 6-formylamino-5-methoxy-1H-indole-2-carboxylate; step e, treating ethyl 6-formylamino-5-methoxy-1H-indole-2-carboxylate with acetone in the presence of hydrochloric acid and water to form ethyl 6-amino-5-methoxy-1H-indole-2-carboxylate; step f, treating ethyl 6-amino-5-methoxy-1H-indole-2-carboxylate with dimethyl 2-oxoglutaconate in the presence of methylene chloride to form 9-hydroxy-5-methoxy-6,7,8,9-tetrahydro-1H-pyrrolo[2,3-f]quinoline-2,7,9-tricarboxylic acid 2-ethyl ester 7,9-dimethyl ester; step g, treating 9-hydroxy-5-methoxy-6,7,8,9-tetrahydro-1H-pyrrolo[2,3-f]quinoline-2,7,9-tricarboxylic acid 2-ethyl ester 7,9-dimethyl ester with copper (II) acetate monohydrate in the presence of methylene chloride and hydrogen chloride to form 5-methoxy-1H-pyrrolo[2,3-f]quinoline-2,7,9-tricarboxylic acid 2-ethyl ester 7,9-dimethyl ester; step h, treating 5-methoxy-1H-pyrrolo[2,3-f]quinoline-2,7,9-tricarboxylic acid 2-ethyl ester 7,9-dimethyl ester with cerric ammonium nitrate in the presence of acetonitrile and water to form 4,5-dioxo-4,5-dihydro-1H-pyrrolo[2,3-f]quinoline-2,7,9-tricarboxylic acid 2-ethyl ester 7,9-dimethyl ester; step i, treating 4,5-dioxo-4,5-dihydro-1H-pyrrolo[2,3-f]quinoline-2,7,9-tricarboxylic acid 2-ethyl ester 7,9-dimethyl ester in tetrahydrofuran with lithium hydroxide at low temperature, adding halide salt, followed by adding hydrochloric acid to form a triacid salt comprised of a single metal; step j, dissolving said triacid salt in sulfuric acid and adding the resulting acidic solution to water to form 4,5-dioxo-4,5-dihydro-1H-pyrrolo[2,3-f]quinoline-2,7,9-tricarboxylic acid (PQQ).

In a preferred embodiment, the temperature of step i is maintained at or below 17° C. In a more preferred embodiment, the temperature of step i is maintained at 16-17° C.

In one embodiment, the pH of step i is maintained at <6. In a preferred embodiment, the pH is maintained at 5.3.

In a preferred embodiment, the order of addition of reagents at step i is: 4,5-dioxo-4,5-dihydro-1H-pyrrolo[2,3-f]quinoline-2,7,9-tricarboxylic acid 2-ethyl ester 7,9-dimethyl ester, tetrahydrofuran, lithium hydroxide, potassium chloride, and hydrochloric acid.

In a preferred embodiment, the triacid salt formed in step i is according to Formula I: 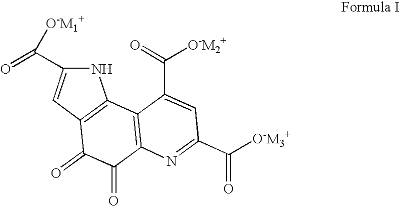
where M₁ is hydrogen or potassium; M₂ is hydrogen or potassium; M₃ is hydrogen or potassium; provided that at least one of M₁, M₂, and M₃ is not hydrogen. In another embodiment, at least two of M₁, M₂, and M₃ are not hydrogen. In a another embodiment, M₁, M₂, and M₃ are all potassium.

In a preferred embodiment, a triacid is created by the following: step a, treating 2-methoxy-5-nitroaniline with formic acid in the presence of acetic acid and water to form N-(2-methoxy-5-nitrophenyl)formamide; step b, treating N-(2-methoxy-5-nitrophenyl)formamide with hydrogen in the presence of palladium and dimethyl formamide to form N-(5-amino-2-methoxyphenyl)formamide; step c, treating N-(5-amino-2-methoxyphenyl)formamide with sodium nitrite and fluoroboric acid in the presence of water and ethanol, followed by ethyl 2-methylacetoacetate and sodium acetate in the presence of water to form ethyl 2-[(3-formylamino-4-methoxyphenyl)hydrazono]-propionate; step d, treating ethyl 2-[(3-formylamino-4-methoxyphenyl)hydrazono]-propionate with formic acid to form ethyl 6-formylamino-5-methoxy-1H-indole-2-carboxylate; step e, treating ethyl 6-formylamino-5-methoxy-1H-indole-2-carboxylate with acetone in the presence of hydrochloric acid and water to form ethyl 6-amino-5-methoxy-1H-indole-2-carboxylate; step f, treating ethyl 6-amino-5-methoxy-1H-indole-2-carboxylate with dimethyl 2-oxoglutaconate in the presence of methylene chloride to form 9-hydroxy-5-methoxy-6,7,8,9-tetrahydro-1H-pyrrolo[2,3-f]quinoline-2,7,9-tricarboxylic acid 2-ethyl ester 7,9-dimethyl ester; step g, treating 9-hydroxy-5-methoxy-6,7,8,9-tetrahydro-1H-pyrrolo[2,3-f]quinoline-2,7,9-tricarboxylic acid 2-ethyl ester 7,9-dimethyl ester with copper (II) acetate monohydrate in the presence of methylene chloride and hydrogen chloride to form 5-methoxy-1H-pyrrolo[2,3-f]quinoline-2,7,9-tricarboxylic acid 2-ethyl ester 7,9-dimethyl ester; step h, treating 5-methoxy-1H-pyrrolo[2,3-f]quinoline-2,7,9-tricarboxylic acid 2-ethyl ester 7,9-dimethyl ester with cerric ammonium nitrate in the presence of acetonitrile and water to form 4,5-dioxo-4,5-dihydro-1H-pyrrolo[2,3-f]quinoline-2,7,9-tricarboxylic acid 2-ethyl ester 7,9-dimethyl ester; step i, treating 4,5-dioxo-4,5-dihydro-1H-pyrrolo[2,3-f]quinoline-2,7,9-tricarboxylic acid 2-ethyl ester 7,9-dimethyl ester in tetrahydrofuran with lithium hydroxide at low temperature, adding excess salt, followed by adding hydrochloric acid. In a more preferred embodiment, the salt added in step i is potassium chloride.

The above description sets forth generally the more important features of the present invention in order that the detailed description thereof that follows may be understood, and in order that the present contributions to the art may be better appreciated. Other objects and features in the present invention will become apparent from the following detailed description considered in conjunction with the accompanying drawings. It is to be understood, however, that the drawings are designed solely for the purpose of illustration and not as a definition of the limits of the invention, for which reference should be made to the amended claims.

### BRIEF DESCRIPTION OF THE DRAWINGS

FIG. 1 is a schematic diagram that generally depicts the overall synthetic process for the production of PQQ (1);

FIG. 2 is a schematic diagram that generally depicts the synthetic process of the final step of the present invention for the production of PQQ (1);

FIG. 3A is a ¹HNMR spectrum of PQQ (1);

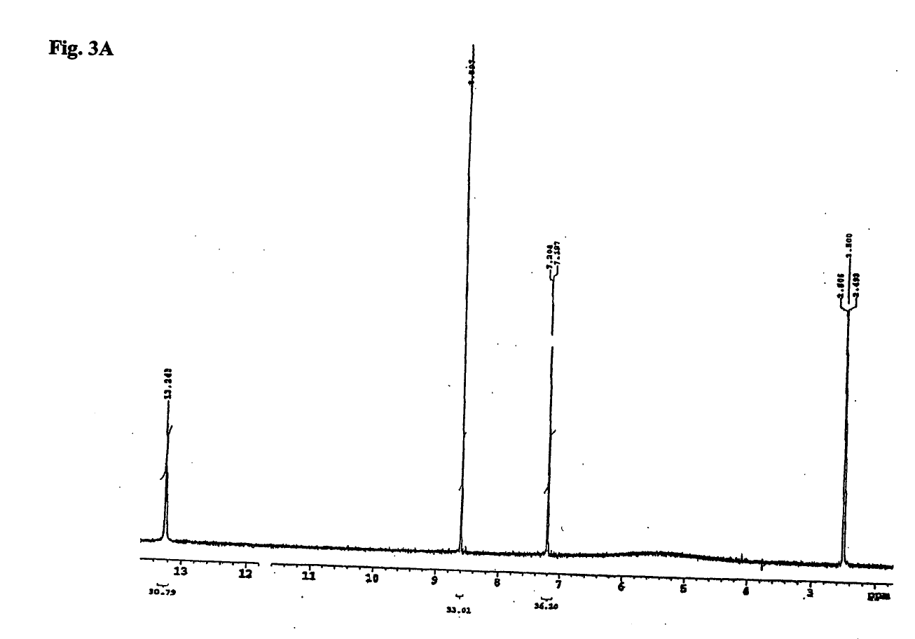

FIG. 3B is a ¹³CNMR spectrum of PQQ (1); and

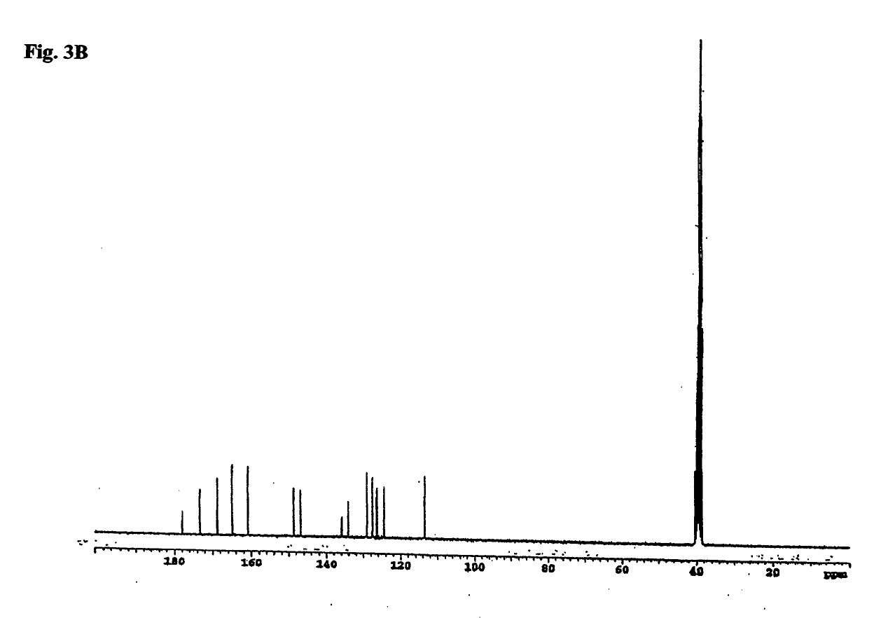

FIG. 3C is a HPLC chromatogram of PQQ (1) (98.9%) at 255 nm.

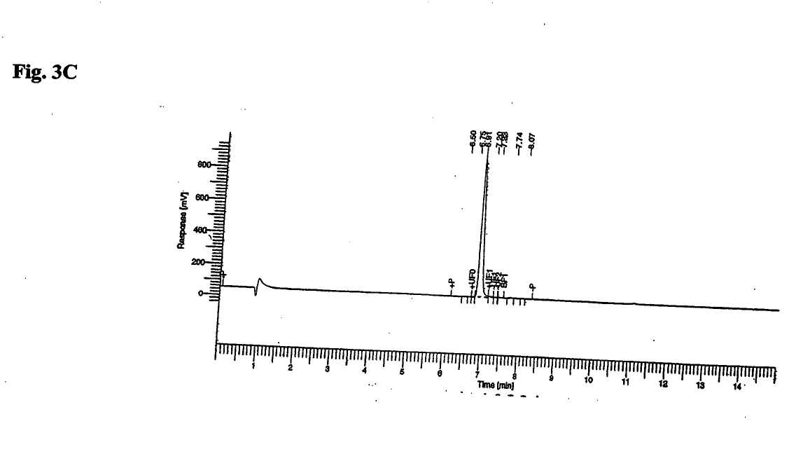

### DETAILED DESCRIPTION OF THE INVENTION

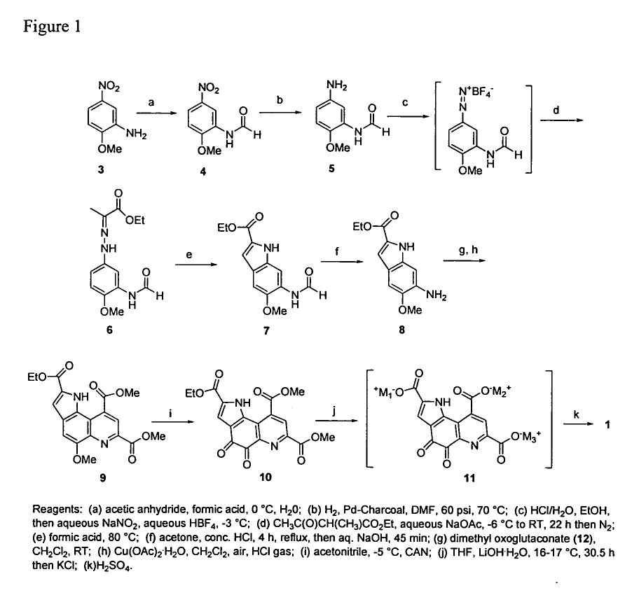

PQQ (1) was first described in Salisbury et al., 1979, Nature, 280:843-844. The synthesis of 1, in accordance with the present invention involves a nine step linear synthesis (FIG. 1). This synthesis was used, e.g. to produce 17 g of 1 in 14% overall yield.

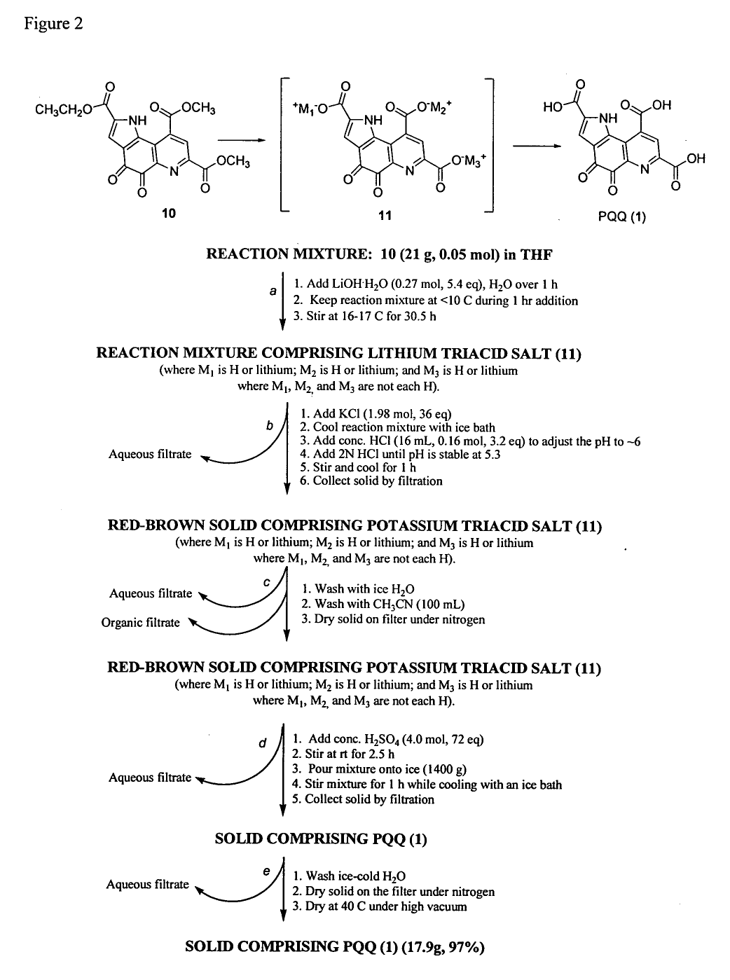

As illustrated in FIG. 2, in the final step of the method of the present invention, 4,5-dioxo-4,5-dihydro-1H-pyrrolo[2,3-f]quinoline-2,7,9-tricarboxylic acid 2-ethyl ester 7,9-dimethyl ester (10) was reacted with lithium hydroxide (LiOH) in tetrahydrofuran-water (THF—H₂O) at low temperature to produce the crude lithium triacid salt in solution (procedure a). Unlike the synthesis of Martin, the halide salt, potassium chloride (KCl), was added before the pH of the reaction mixture was adjusted to 5.3 using hydrochloric acid (HCl) (procedure b). The resulting triacid potassium salt, 11, precipitated out of solution under conditions of carefully controlled pH (5.3), was collected by vacuum filtration and washed with water and solvent (procedure c). The triacid potassium salt was converted to PQQ by dissolving 11 in concentrated sulfuric acid (H₂SO₄) and pouring the resulting acid solution onto ice to afford the final product, PQQ (1) (procedures d and e).

The following techniques were used to characterize the product and to detect impurities: thin layer chromatography (TLC), nuclear magnetic resonance spectroscopy (NMR), high pressure liquid chromatography (HPLC), and elemental analysis. PQQ was further purified and impurities removed by additional treatment with sulfuric acid, isolation of PQQ by filtration, and drying under vacuum.

The total synthetic process for preparing PQQ (1), began with the two step preparation of dimethyl 2-oxoglutaconate (12). The diketone 12 is used later as a reagent in step g (the seventh step) of the PQQ (1) total synthesis as shown in FIG. 1. One preferred method for the preparation of the diketone 12 is according to the method of Corey (Corey et al., J. Am. Chem. Soc., 103 (1981) 5599). Dimethyl 2-oxoglutarate and methylene chloride (CH₂Cl₂) were combined and heated to reflux, followed by the addition of a solution of bromine and CH₂Cl₂ which was stirred at reflux for 3.5 hours. The reaction mixture was cooled and then concentrated in vacuo using a rotary evacuator. The residue was taken up in diethyl ethyl ether (Et₂O), and triethylamine (Et₃N) was added slowly keeping the temperature below 35° C. After stirring for 45 minutes, the triethylamine bromide by-product was filtered off and nitrogen was bubbled through the filtrate for 2 hours. The filtrate was vacuum filtered through silica gel and the silica gel was washed with Et₂O. The combined ethereal solutions were concentrated in vacuo using a rotary evacuator to produce dimethyl 2-oxoglutaconate (12).

As shown in FIG. 1, the linear PQQ synthesis began with the addition of commercially available (Aldrich, Milwaukee, Wis.) 2-methoxy-5-nitroaniline (3) to a cooled solution of acetic anhydride (Ac₂O) and formic acid to afford a thick slurry, which was stirred overnight. Water was added to the reaction mixture which was then stirred for an additional two days. The pale product was collected by vacuum filtration and washed with water until the pH of the washing solution was neutral. This first step of the synthesis afforded after drying on the house vacuum, N-(2-methoxy-5-nitrophenyl)formamide (4).

In step b, a Parr™ pressure reactor was charged with N-(2-methoxy-5-nitrophenyl)formamide (4), dimethylformamide (DMF) and 5% palladium on charcoal. The reactor was pressurized with hydrogen, and the hydrogenation was conducted at elevated temperature. The reaction was exothermic and required cooling. The catalyst was removed by vacuum filtration of the mixture through Celite™ and the filtrate was concentrated in vacuo. The residue was taken up in methanol (MeOH) and stirred overnight. The slurry was cooled in an ice bath and stirred, and the product was collected by filtration and washed with ether (Et₂O) to afford N-(5-Amino-2-methoxyphenyl)formamide (5).

Next, in step c, a reactor was charged with concentrated hydrochloric acid (HCl) and water and cooled to −26° C. N-(5-amino-2-methoxyphenyl)formamide (5) was added to the acid mixture followed by the addition of ethanol. Next, a solution of aqueous sodium nitrite (NaNO₂) was added and the temperature of the reaction mixture was held at −20 to −25° C. following the addition. Ethanol cooled at 0° C. was added and stirring was continued for 20 minutes. Fluoroboric acid (HBF₄) was added, and the low temperature was maintained during the addition. Ethanol was added and stirring was continued for another 30 minutes. The reaction mixture was allowed to warm over 30 minutes. The diazonium tetrafluoroborate salt was collected by filtration and washed with cold ethanol. A slurry of the salt in ethanol was stirred at low temperature and a solution of ethyl 2-methylacetoacetate (CH₃C(O)CH(CH₃)CO₂Et) (Aldrich, Milwaukee, Wis.), sodium acetate (NaOAc) and water was added while maintaining a low temperature (step d). The cooling bath was removed and stirring was continued overnight. Nitrogen was bubbled through the mixture overnight, and the product was collected by vacuum filtration and washed with a mixture of ethanol/water. In this reaction, the initial substitution product underwent spontaneous deacetylation and double bond migration, a process known as the Japp-Klingemann Reaction. The resulting solid was dried under vacuum on the filter overnight, washed with additional ethanol and isopropyl alcohol to afford ethyl 2-[(3-formylamino-4-methoxyphenyl)hydrazono]-propionate (6).

Following in step e, ethyl 2-[(3-formylamino-4-methoxyphenyl)hydrazono]-propionate (6) and formic acid were placed in a reaction vessel and stirred overnight at 80° C. The reaction mixture was allowed to cool and ethanol was added. The bright-green slurry was cooled to 0° C. and stirred. The product was collected by vacuum filtration, washed with ethanol, dried on a filter and dried at 80° C. under vacuum to afford the desired indole, ethyl 6-formylamino-5-methoxy-1H-indole-2-carboxylate (7) via the Fischer Indole Synthesis.

To a solution containing acetone, concentrated HCl and water was added ethyl 6-formylamino-5-methoxy-1H-indole-2-carboxylate (7). The reaction mixture was refluxed for four hours and then cooled to 0° C. (step f). The resulting solid was collected by vacuum filtration and left to dry under vacuum on the filter overnight. The dried material was taken up and stirred in 1.5N aqueous sodium hydroxide (NaOH) for 45 minutes. The product was collected by vacuum filtration and washed to afford ethyl 6-amino-5-methoxy-1H-indole-2-carboxylate (8).

To a reaction vessel charged with ethyl 6-amino-5-methoxy-1H-indole-2-carboxylate (8) and CH₂Cl₂ was added a solution of dimethyl 2-oxoglutaconate (12) in CH₂Cl₂ (step g). At the end of the addition, the reaction mixture was transferred to a second reaction vessel, stirred in cold water, and vacuum filtered. The product was collected, washed with CH₂Cl₂-heptane, and dried under vacuum to afford 9-hydroxy-5-methoxy-6,7,8,9-tetrahydro-1H-pyrrolo[2,3-f]quinoline-2,7,9-tricarboxylic acid 2-ethyl ester 7,9-dimethyl ester which was combined with copper acetate monohydrate (Cu(OAc)₂H₂O) and CH₂Cl₂ (step h). The reaction mixture was stirred with a stream of air and anhydrous HCl bubbling through while it was kept at room temperature. Bubbling was continued for six hours and then bubbling of only air was continued overnight. The reaction mixture was quenched by the addition of an aqueous sodium carbonate (Na₂CO₃) solution. The reaction mixture was stirred for two hours and vacuum filtered. Methylene chloride was used to further dilute the resulting filtrate. The mixture was stirred to ensure that the product was completely dissolved in solution. The CH₂Cl₂ layer was separated and the aqueous layer was extracted with CH₂Cl₂. The organic layers were combined, washed with H₂O, dried over Na₂SO₄, filtered and concentrated to afford a solid which was stirred overnight with Et₂O, cooled in an ice bath, filtered, washed with Et₂O and dried at 50° C. under high vacuum to afford 5-methoxy-1H-pyrrolo[2,3-f]quinoline-2,7,9-tricarboxylic acid 2-ethyl ester 7,9-dimethyl ester (9).

In step i, 5-methoxy-1H-pyrrolo[2,3-f]quinoline-2,7,9-tricarboxylic acid 2-ethyl ester 7,9-dimethyl ester (9) and acetonitrile were placed in a reaction vessel, cooled and stirred. To this suspension was added a solution of aqueous cerric ammonium nitrate ((NH₄)₂Ce(NO₃)₆ or CAN) to produce a bright orange solution which was then poured into cold water. The cerrium salts were collected under vacuum and the filtrate was extracted with CH₂Cl₂. The organic extracts were dried over magnesium sulfate (MgSO₄) and concentrated. The resulting residue was taken up in a solution of toluene-ethyl acetate (EtOAc), and the resulting crystals were collected by filtration, washed with EtOAc-heptane, taken up in CH₂Cl₂ and stirred with silica gel for 1 hour, and then filtered through Celite™. The solvent was removed to produce 4,5-dioxo-4,5-dihydro-1H-pyrrolo [2,3-f]quinoline-2,7,9-tricarboxylic acid 2-ethyl ester 7,9-dimethyl ester (10).

As shown in the final steps j and k in FIG. 1, and as illustrated more detail in FIG. 2, 4,5-dioxo-4,5-dihydro-1H-pyrrolo [2,3-f]quinoline-2,7,9-tricarboxylic acid 2-ethyl ester 7,9-dimethyl ester (10) and THF were combined in a reaction vessel and an aqueous solution of a base, such as LiOH, NaOH, KOH, or CsOH, was added keeping the temperature of the reaction mixture below 10° C. In a preferred embodiment, LiOH was used to form the lithium triacid salt (i.e. one lithium ion for each acid group). The reaction mixture was stirred at 16-17° C. for 30.5 hours (procedure a). Small scale hydrolysis reactions were conducted to determine whether varying the temperature had an effect on the amount of impurity produced. The percentage of impurity was found to increase as the temperature of the reaction was increased. The optimal reaction temperature was determined to be 16-17° C.

In procedure b, after stirring, a large excess of salt e.g., KCl, NH₄Cl, (NH₄)₂CO₃ or K₂CO₃ was added to the reaction mixture which was then cooled in an ice bath. In a preferred embodiment, KCl was added. The reaction mixture was acidified using a mineral acid, such as HCl, H₂SO₄, or formic acid until the pH of the reaction mixture was adjusted to 6. In a preferred embodiment, HCl was used to adjust the pH. The pH was then more accurately adjusted to 5.3 using 2N HCl. Careful control of the pH allowed the triacid salt to be isolated in consistent yield and excellent purity from batch to batch. In a preferred embodiment, the triacid salt is comprised of a single counterion (e.g., potassium (K⁺)). In another embodiment, the triacid salt is comprised of one of the following metals: potassium, cesium, ammonium, or sodium. In a preferred embodiment, the metal is potassium, thus producing the triacid salt 11 (as shown below). Using this procedure to produce the potassium salt under careful pH control, the majority of the organic impurities remained in solution and the triacid was isolated with minimal impurities. Prior art studies did not evaluate the effect of pH on the purity of triacid isolated or the amount of reaction impurities formed. See e.g., Martin et. al. 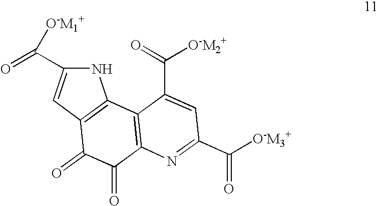
where
M₁ is hydrogen or potassium; M₂ is hydrogen or potassium; M₃ is hydrogen or potassium; where M₁, M₂, and M₃ are not each hydrogen.

After the addition of acid, the reaction mixture was cooled, and then the resulting solid was collected by vacuum filtration, washed with ice water and acetonitrile, and dried (step c). The triacid potassium salt 11 was dissolved in concentrated sulfuric acid (H₂SO₄) and stirred for 2.5 hours (step d). The acid solution was poured onto ice and the resulting suspension of dark solids was stirred. The solid product was collected by filtration, washed with ice cold water, dried under nitrogen and vacuum to afford several grams of PQQ (1) with a barely detectable amount of impurity.

PQQ was further purified in a large batch by dissolving several grams of the material (e.g., between 1-100 g e.g., 50-100 g, e.g., 75-100 g) in concentrated H₂SO₄ and stirring at, or below, room temperature. Sulfuric acid is unique in its ability to dissolve PQQ which makes it an appropriate solvent to use for purification. The acid solution was added dropwise to 5 L of water, keeping the temperature at <33° C. The desired product PQQ, precipitated from the solution upon stirring at room temperature, was collected by filtration and washed with water, and dried under vacuum.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

### Example 1

### Synthesis of PQQ (1)

A. Dimethyl 2-oxoglutaconate (12)

Dimethyl 2-oxoglutarate (87.0 g, 0.50 mol) and 328 mL of methylene chloride were placed in a 1-L, 3-neck flask equipped with a mechanical stirrer, an addition funnel and a heating mantle. The solution was stirred and brought to reflux. A solution of bromine (77.0 g, 0.48 mol) in 165 mL of methylene chloride was added over a 45-min period. The reaction mixture was stirred and refluxed for an additional 3.5 hours.

The reaction mixture was concentrated in vacuo to afford an orange oil. Ethyl ether (328 mL) was added and removed in vacuo. Additional ethyl ether (328 mL) was added, the solution was stirred, and triethylamine (65.6 mL, 0.44 mol) was slowly added with the temperature held below 35° C. The heavy slurry was stirred for 45 min, and the triethylamine hydrobromide was filtered off and washed with ethyl ether. Nitrogen was bubbled through the filtrate for two hours. About 700 mL of a dark-red, opaque solution remained. The solution was filtered through 70 g of silica gel. The silica gel was washed with 3.5 L of ethyl ether. The combined ethyl ether solution was concentrated in vacuo to afford a bright yellow solid, which, after drying at room temperature afforded 71.5 g (83.1%) of the title compound.

B. N-(2-methoxy-5-nitrophenyl)formamide (4)

Into a 5-L, 3-neck flask equipped with a mechanical stirrer, a temperature probe and an ice bath was placed, acetic anhydride (367 g, 9.72 mol). Formic acid (367 g, 6.01 mol) was added with stirring and cooling. The rate of addition was such that the temperature did not exceed 32° C. Stirring was continued for one hour at ambient temperature. Cooling was again applied and 2-methoxy-5-nitroaniline (3) (415 g, 2.47 mol) was added in portions over a 1.5-hour period. The temperature was kept below 32° C. during addition. The thick-yellow slurry was stirred overnight.

Water (3 L) was added and the mixture was stirred for an additional 48 hours. The yellow product was collected by filtration and washed with water until the pH of the aqueous washing was neutral. A total of 9.2 L of water was used. The product was dried at 60° C. under house vacuum and afforded 480 g (99.1%) of the title compound.

C. N-(5-Amino-2-methoxyphenyl)formamide (5)

A 2-L Parr pressure reactor was used for this reaction. It was equipped with a mechanical stirrer, a temperature sensor, a cooling coil, and a heating mantle. N-(2-methoxy-5-nitrophenyl)formamide (4) (170.0 g, 0.867 mol), 1100 mL of DMF, and 7.75 g of 5% palladium on charcoal were placed in the reactor. Air was purged from the reactor by pressurizing the reactor to 60 psig with nitrogen and venting. The reactor was then pressurized to 60 psig with hydrogen and vented three times.

Hydrogenation was conducted at 70° C. and 60 psi of hydrogen. Frequent recharging with hydrogen was required. This was normally done when the pressure had dropped to 30 psi. The reaction was quite exothermic and required cooling by passing water through the cooling coil until the reaction was nearly complete. Hydrogen uptake slowed dramatically after three hours and the reaction was stopped.

The palladium on charcoal catalyst was removed by filtration of the reaction mixture through Celite™ 521. The solvent was removed in vacuo. The residue was combined with the unpurified products of two other reactions identical to the above reaction and each reaction was conducted on 170 g scale (3 total) and stirred overnight with 300 mL of methanol. The dark brown slurry was cooled in an ice bath and stirred for an additional three hours. The product was collected by filtration and washed with ethyl ether. The filtrates resulting from the initial washings were green. Washing with ethyl ether was continued until the filtrate was light in color. The yield of the title compound was 387.9 g (89.7%).

D. Ethyl 2-[(3-formylamino-4-methoxyphenyl)hydrazono]-propionate (6)

Into a 12-L reactor equipped with a mechanical stirrer, a temperature probe and a dry ice bath were placed, 560 mL of conc hydrochloric acid and 105 mL of water. The solution was stirred and cooled to −26° C. N-(5-Amino-2-methoxyphenyl)formamide (5) (368.0 g, 2.21 mol) was added in portions over a 15-min period. Ethanol (81 mL) was added. Then a solution of sodium nitrite (171.3 g, 2.48 mol) in 257 mL of water was added over a 25-min period. The temperature of the reaction mixture was held at −20 to −25° C. during the addition. Stirring with cooling was continued for 15 min and 1165 mL of ice-cold ethanol was added. Stirring was continued for 20 min with the temperature held at −5 to −10° C.

Fluoroboric acid (370 mL 50% aq. 2.96 mol) was added over a 10 min period. The temperature was held below −3° C. during the addition. Ethanol (739 mL) was added and stirring at −5° C. was continued for 30 min and then the reaction mixture was allowed to warm to 5° C. over a 30 min period. The bright yellow-tan slurry became darker as it warmed. The diazonium tetrafluorborate salt was collected by filtration and washed with cold ethanol until the washings were light colored.

The diazonium tetrafluorborate salt was transferred back to the reactor and stirred with 2030 mL of cold ethanol. The reaction mixture was cooled to −8° C. A solution of ethyl 2-methylacetoacetate (314.5 g, 2.18 mol), sodium acetate (602.4 g, 7.34 mol), and 1770 mL of water was added over a 12 min period. The temperature was kept below −6° C. during the addition. The cooling bath was removed and stirring was continued for 22 hours.

Nitrogen was then bubbled through the reaction mixture overnight. The product was collected by filtration and washed with 740 mL of 10% ethanol/water and with 5.5 L of cold water. The wet cake was dried on the filter overnight and then washed with 800 mL of cold ethanol. The resulting solid was dried at 50° C. under house vacuum and afforded 407.5 g (65.9%) of the title compound.

Further concentration of the mother liquors by bubbling nitrogen through the resulting mother liquors for two days gave additional solid material, which was collected by filtration. The additional solid material was washed with 500 mL of 10% ethanol/water, 2 L of cold water, and 500 mL of isopropyl alcohol to yield an additional 13.3 g (3.6%) of the title compound. The total yield of the title compound was 420.8 g (69.5%).

E. Ethyl 6-formylamino-5-methoxy-1H-indole-2-carboxylate (7)

Ethyl 2-[(3-formylamino-4-methoxyphenyl)hydrazono)propionate (6) (425.0 g, 1.53 mol) and 1530 mL of formic acid were placed in a 3-L, 3-neck flask equipped with a mechanical stirrer, a temperature probe, a condenser, and a heating mantle. The reaction mixture was stirred overnight at 80° C.

The reaction mixture was allowed to cool and 770 mL of ethanol was added. The bright-green slurry was cooled to 0° C. and stirred for two hours. The product was collected by filtration and washed with 700 mL of ethanol. The green-brown product was dried on the filter and then at 80° C. under house vacuum to afford 300.1 g (75.2%) of the title compound.

F. Ethyl 6-amino-5-methoxy-1H-indole-2-carboxylate (8)

Into a 12-L, 3-neck flask equipped with a mechanical stirrer, a temperature probe, a heating mantle, and a condenser were placed, 7.70 L of acetone and a solution of 365 mL of conc. hydrochloric acid diluted with water to 765 mL. The solution was stirred at room temperature and ethyl 6-formamido-5-methoxy-1H-indole-2-carboxylate (7) (300.1 g, 1.14 mol) was added to give a tan-green slurry. The reaction mixture was refluxed for 4 hours and then cooled to 0° C. in an ice bath. The resulting tan product was collected by filtration and dried on the filter overnight. The dried product was broken up and stirred with 1.70 L of 1.5N aqueous sodium hydroxide for 45 min. The product was collected by filtration and washed with 6 times with 2 L of cold water. Washing took 5.5 hours and the final washes had a neutral pH to afford 214.0 g (79.8%) of the title compound.

G. 5-Methoxy-1H-pyrrolo[2,3-f]quinoline-2,7,9-tricarboxylic acid 2-ethyl ester 7,9-dimethyl ester (9)

Into a 3-L, 3-neck flask equipped with a mechanical stirrer, an addition funnel, a temperature probe, a heating mantle, and a condenser vented to a nitrogen line under static pressure were placed, ethyl 6-amino-5-methoxy-1H-indole-2-carboxylate (8) (213.6 g, 0.912 mol) and 1325 mL of methylene chloride. The green suspension was stirred under nitrogen and a solution of dimethyl 2-oxoglutaconate (12) (175.0 g, 1.02 mol) in 700 mL of methylene chloride was added at room temperature over a 10-min period. An additional 100 mL of methylene chloride was used to rinse all of 12 into the reactor. At the end of the addition, the reaction mixture was nearly black. There was no rise in temperature. The reaction mixture was stirred overnight as a matter of convenience.

The reaction mixture was transferred to a 3-L, 1-neck flask and concentrated in vacuo to about one fourth of its original volume. The reaction mixture was then stirred and cooled in cold water for 1 hour. The final temperature was 7° C. The product was collected by filtration and washed with 350 mL of 1:5 methylene chloride—heptane. The amber product was partially dried on the filter and then at 50° C. under high vacuum to afford 270.8 g (73.1%) of the 9-hydroxy-5-methoxy-6,7,8,9-tetrahydro-1H-pyrrolo[2,3-f]quinoline-2,7,9-tricarboxylic acid 2-ethyl ester 7,9-dimethyl ester.

Into a 22-L, jacketed reactor equipped with a mechanical stirrer, a bottom-drop valve, an addition funnel, a temperature probe, a heating mantle, a dip tube and a condenser were placed, 9-hydroxy-5-methoxy-6,7,8,9-tetrahydro-1H-pyrrolo[2,3-f]quinoline-2,7,9-tricarboxylic acid 2-ethyl ester 7,9-dimethyl ester (270.8 g, 0.666 mol), Cu(OAc)₂—H₂O (146.3 g, 0.733 mol), and 11.50 L of methylene chloride. The reaction mixture was stirred and a stream of air and anhydrous hydrogen chloride were bubbled through the reaction mixture. The reaction was exothermic and the reactor jacket temperature was set for 10° C. to maintain the reaction temperature at about 20° C. After about 30 min, the reaction mixture became less exothermic and the jacket temperature was set to 20° C. Bubbling of air and hydrogen chloride was continued for 6 hours. Bubbling of air through the reaction mixture was continued overnight.

The reaction was quenched by addition of a solution of 550 g of sodium bicarbonate in 6.0 L of water over a 30-min period. Very little evolution of carbon dioxide was noted. A very dark blue solution and blue-green solids resulted. The demarcation between the organic and aqueous phases was poor. The mixture was stirred 2 hours and filtered. Filtration was slow taking about 7 hours. Evaporation of methylene chloride left black solids in and at the exit of the filter. Near the end, the slimy, essentially all aqueous mixture was poured into a clean filter and allowed to filter. Both filters were filled with methylene chloride and allowed to drain by gravity overnight. The filters were further rinsed with methylene chloride and the combined filtrate was returned to the reaction vessel. Several liters of methylene chloride were added to bring the volume back to approximately the original volume.

The two-phase mixture was stirred 5 hours to assure that the product was completely in solution. The methylene chloride layer was separated. There were blue-green solids floating in the upper part of the methylene chloride layer. The upper part of the methylene chloride layer was collected separately and the solids were removed by filtration. The aqueous layer was extracted with 2 L of methylene chloride. The combined methylene chloride solution was washed with 4 L of water and dried by stirring with 750 g of sodium sulfate. The solution was filtered and stripped to a black semi-solid with yellow crystalline highlights. The material was stirred overnight with 1 L of ethyl ether. The slurry was cooled in ice and the product was collected by filtration. The product was then washed with 700 mL of ethyl ether and dried at 50° C. under high vacuum to afford 221.7 g (86.1%) of the title compound (9) as a brassy colored product.

I. 4,5-Dioxo-4,5-dihydro-1H-pyrrolo[2,3-f]quinoline-2,7,9-tricarboxylic acid 2-ethyl ester 7,9-dimethyl ester (10)

Into a 3-L, 3-neck flask equipped with a mechanical stirrer, a temperature probe, an addition funnel, and a dry ice/acetone bath were placed 5-methoxy-1H pyrrolo[2,3-f]quinoline-2,7,9-tricarboxylic acid 2-ethyl ester 7,9-dimethyl ester (9) (12.09 g, 0.0313 mol) and 600 mL of acetonitrile. The suspension was stirred and cooled to −5° C. A solution of cerric ammonium nitrate (84.87 g, 0.155 mol) in 120 mL of water was added over a five-minute period. A bright orange solution resulted. Stirring and cooling at −5° C. was continued for 1.5 hours.

The reaction mixture was poured into 1380 mL of vigorously-stirred, cold water and stirring was continued for 0.5 hour. The cerrium salts were filtered off and the filtrate was extracted with 3 times with 300 mL of methylene chloride. The methylene chloride solution was dried over 75 g of magnesium sulfate and the solvent removed in vacuo to produce 10.15 g of solid. The resulting solid was stirred in a solution of 10 mL of toluene and 10 mL of ethyl acetate. The resulting bright red-orange crystals were collected by filtration and washed with a solution of 3:1 ethyl acetate—heptane. The crystals were taken up in 360 mL of methylene chloride and stirred with 5 g of silica gel for one hour. The solution was filtered through Celite™ 521 and stripped to 7.50 g (62.0%) of the title compound as a red-orange solid.

J. 4,5-Dioxo-4,5-dihydro-1H-pyrrolo[2,3-f]quinoline-2,7,9-tricarboxylic Acid (PQQ) (1)

Into a 1-L, 3-neck flask equipped with a mechanical stirrer, a temperature probe, an addition funnel, a nitrogen purge system, and an ice bath were placed, 4,5-dioxo-4,5-dihydro-1H-pyrrolo[2,3-f]quinoline-2,7,9-tricarboxylic acid 2-ethyl ester 7,9-dimethyl ester (10) (21.45 g, 0.0555 mol) and 215 mL of THF. The heterogeneous solution was stirred under nitrogen and a solution of lithium hydroxide monohydrate (11.58 g, 0.276 mol) in 515 mL of water was added over a one hour period. The temperature was held below 10° C. during the addition. The reaction mixture was stirred at 16-17° C. for 30.5 hours.

Potassium chloride (15 g, 1.98 mol) was added. The flask was iced down and the pH of the reaction mixture was adjusted to approximately 6 by addition of 10.0 mL of conc. hydrochloric acid. The pH was further adjusted to 5.3 by addition of 2 N hydrochloric acid. Successive additions were required to attain a stable pH. A total of 16.0 mL of 2N hydrochloric acid was added. Stirring and cooling were continued for one hour. The red-brown solid that formed was collected by filtration, washed with a small amount of ice-water and with 100 mL of acetonitrile. The resulting red-brown triacid salt 11 was dried on the filter under a stream of nitrogen.

Dried 11 (21.21 g) was dissolved in 330 mL of concentrated sulfuric acid and stirred for 2.5 hours. The acid solution was poured onto 1400 g of ice to yield a suspension of blood-red solids. The suspension was stirred with cooling for one hour. The product was collected by filtration and washed with ice-cold water. It was dried on a filter under a stream of nitrogen and then at 40° C. under high vacuum to afford 17.85 g (97.4%) of the title compound. The NMR spectra indicated the presence of water even though it had been dried to near constant weight. Thermal gravimetric analysis gave an ash of 7.8%. Thermal gravimetric analysis involves heating the sample under a stream of oxygen. Any ash that remains after heating is indicative of metal (inorganic) impurities.

### Example 2

### Batch Purification of PQQ (1)

A large batch of PQQ (1) (75.49 g) was additionally purified to remove any residual impurity by dissolving PQQ in 100 mL of concentrated sulfuric acid. The suspension was stirred at room temperature for 2 hours. The acid solution was added slowly dropwise to 5 L of vigorously stirred water over a 40 min period while keeping the temperature at <33° C. The desired product precipitated from the solution and the suspension was stirred at room temperature for one hour. The product was collected by filtration and washed with 1 L of water. The product was dried at 40° C. under high vacuum. The recovery was 63.0 g (83.5%). After accounting for purity and two additional purifications, the yield of PQQ was 71.6%.

### Example 3

### Characterization of 4,5-dioxo-4,5-dihydro-1H-pyrrolo[2,3-f]quinoline-2,7,9-tricarboxylic Acid, PQQ (1)

Routine procedures were used for purity analysis and characterization of PPQ (1) (e.g., NMR, HPLC, Karl Fisher titration, Elemental Analysis). Table 1 shows a summary of the analysis results for a batch of PQQ produced on a 62.7 g scale. ¹HNMR data reported in literature was used for comparison with data obtained using the present invention (Table 2). For HPLC analysis, a solvent gradient comprised of buffer (20 mM KH₂PO₄ (pH 2.1)) and acetonitrile (CH₃CN) was applied. The gradient was as follows: T=0, 2:98 CH₃CN/buffer; T=6 min, 90:10 CH₃CN/buffer; T=10 min, 90:10 CH₃CN/buffer; T=16 min, 2:98 CH₃CN/buffer; and T=25, min 2:98 CH₃CN/buffer at a flow rate of 0.7 mL/min. The sample was prepared by dissolving PQQ in DMSO. The column used was a Waters Atlantis™ C18 (SN W40541), 3 μm, 3.0×100 mm. Injection volume was 5 microliters. For liquid chromatography mass spectral analysis, an isocratic solvent system comprised of 50% acetonitrile and 50% buffer (5 mM ammonium acetate in water) was applied at 0.3 mL/min. Run time was 12 min. The sample was prepared by dissolving 1.25 mg of PQQ in 10 mL of DMSO. The resulting solution was further diluted in DMSO 1:50. The injection volume was 10 microliters.

TABLE 1
PQQ Summary of Analysis Results

| Test | <Test Method> | Test Result |
|---|---|---|
| Identity | 1H NMR | Conforms |
| Identity | 13C NMR | Conforms |
| Purity | HPLC TAN | 98.9% |
| Impurity Profile | HPLC TAN | Largest impurity: 0.2% |
| Percent Water | Karl Fisher | 6.4% |
| Composition (Theory | Elemental Analysis | Theory: 47.36% C, |
| includes water and ash) | | 2.42% H, 7.89% N |
| | | Actual: 46.85% C, |
| | | 2.43% H, 7.79% N |
| Loss on heating | TGA | 0.6% ash |

Assigned Purity: 91.9%

TABLE 2
¹HNMR Data for PQQ

| | Reference Value |
|---|---|
| Method of the Invention | (from Martin) |
| ¹HNMR(300MHz, (D₆)DMSO): | ¹HNMR(250MHz, (D₆)DMSO): |
| 7.22(q, J=1 &2, H-C(3)); | 7.21(d, J=2, H-C(3)); |
| 8.60(d, J=1, H-C(8)); | 8.60(s, H-C(S)); 13.25(br, s, NH) |
| 14.40(br, s, NH) | |

### Equivalents

While there have been shown and described and pointed out fundamental novel features of the invention as applied to preferred embodiments thereof, it will be understood that various omissions and substitutions and changes in the form and details of the disclosed invention may be made by those skilled in the art without departing from the spirit of the invention. It is the intention, therefore, to be limited only as indicated by the scope of the claims appended hereto.

What is claimed is:

1. A method for the synthesis of PQQ comprising treating 4,5-dioxo-4,5-dihydro-1H-pyrrolo[2,3-f]quinoline-2,7,9-tricarboxylic acid 2-ethyl ester 7,9-dimethyl ester in tetrahydrofuran with base at low temperature, adding a salt, followed by adding hydrochloric acid.

2. The method of claim 1, wherein the addition of lithium hydroxide and a halide salt forms a triacid salt comprised of a single metal.

3. The method of claim 1, wherein the addition of lithium hydroxide and a carbonate salt forms a triacid salt comprised of a single metal.

4. The method according to claim 1, wherein the temperature of the reaction mixture is maintained at or below 17° C.

5. The method according to claim 1, wherein the temperature of the reaction mixture is maintained at 16-17° C.

6. The method of claim 2, wherein the halide salt is potassium chloride.

7. The method according to claim 2, wherein the metal is potassium.

8. The method according to claim 1, wherein the addition order of the reagents to the reaction flask is: 4,5-dioxo-4,5-dihydro-1H-pyrrolo[2,3-f]quinoline-2,7,9-tricarboxylic acid 2-ethyl ester 7,9-dimethyl ester, tetrahydrofuran, lithium hydroxide, potassium chloride, and hydrochloric acid.

9. The method according to claim 1, wherein the pH is maintained at or below 6.

10. The method according to claim 1, wherein the pH is maintained at 5.3.

11. The method according to claim 2, further comprising the step of dissolving the triacid salt in sulfuric acid and adding the acidic solution to water to form PQQ.

12. A method for the synthesis of PQQ comprising the steps of:
a. treating 2-methoxy-5-nitroaniline with formic acid in the presence of acetic acid and water to form N-(2-methoxy-5-nitrophenyl)formamide;
b. treating N-(2-methoxy-5-nitrophenyl)formamide with hydrogen in the presence of palladium and dimethyl formamide to form N-(5-amino-2-methoxyphenyl)formamide;
c. treating N-(5-amino-2-methoxyphenyl)formamide with sodium nitrite and fluoroboric acid in the presence of water and ethanol, followed by adding ethyl 2-methylacetoacetate and sodium acetate in the presence of water to form ethyl 2-[(3-formylamino-4-methoxyphenyl)hydrazono]-propionate;
d. treating ethyl 2-[(3-formylamino-4-methoxyphenyl)hydrazono]-propionate with formic acid to form ethyl 6-formylamino-5-methoxy-1H-indole-2-carboxylate;
e. treating ethyl 6-formylamino-5-methoxy-1H-indole-2-carboxylate with acetone in the presence of hydrochloric acid and water to form ethyl 6-amino-5-methoxy-1H-indole-2-carboxylate;
f. treating ethyl 6-amino-5-methoxy-1H-indole-2-carboxylate with dimethyl 2-oxoglutaconate in the presence of methylene chloride to form 9-hydroxy-5-methoxy-6,7,8,9-tetrahydro-1H-pyrrolo[2,3-f]quinoline-2,7,9-tricarboxylic acid 2-ethyl ester 7,9-dimethyl ester;
g. treating 9-hydroxy-5-methoxy-6,7,8,9-tetrahydro-1H-pyrrolo[2,3-f]quinoline-2,7,9-tricarboxylic acid 2-ethyl ester 7,9-dimethyl ester with copper (II) acetate monohydrate in the presence of methylene chloride and hydrogen chloride to form 5-methoxy-1H-pyrrolo[2,3-f]quinoline-2,7,9-tricarboxylic acid 2-ethyl ester 7,9-dimethyl ester;
h. treating 5-methoxy-1H-pyrrolo[2,3-f]quinoline-2,7,9-tricarboxylic acid 2-ethyl ester 7,9-dimethyl ester with cerric ammonium nitrate in the presence of acetonitrile and water to form 4,5-dioxo-4,5-dihydro-1H-pyrrolo[2,3-f]quinoline-2,7,9-tricarboxylic acid 2-ethyl ester 7,9-dimethyl ester;
i. treating 4,5-dioxo-4,5-dihydro-1H-pyrrolo[2,3-f]quinoline-2,7,9-tricarboxylic acid 2-ethyl ester 7,9-dimethyl ester in tetrahydrofuran with lithium hydroxide at low temperature, adding a halide salt, followed by adding hydrochloric acid to form a triacid salt comprised of a single metal;
j. dissolving the triacid salt from step i in sulfuric acid and adding the resulting acidic solution to water to form 4,5-dioxo-4,5-dihydro-1 H-pyrrolo[2,3-f]quinoline-2,7,9-tricarboxylic acid (PQQ).

13. The method of claim 12, wherein the temperature of step i is maintained at or below 17° C.

14. The method of claim 12, wherein the temperature of step i is maintained at 16-17° C.

15. The method of claim 12, wherein the pH of step i is maintained at <6.

16. The method of claim 12, wherein the pH of step i is maintained at 5.3.

17. The method of claim 12, wherein the order of addition of reagents at step i is: 4,5-dioxo-4,5-dihydro-1H-pyrrolo[2,3-f]quinoline-2,7,9-tricarboxylic acid 2-ethyl ester 7,9-dimethyl ester, tetrahydrofuran, lithium hydroxide, potassium chloride, and hydrochloric acid.

18. The method of claim 12, forming in step i, the triacid salt according to Formula I: 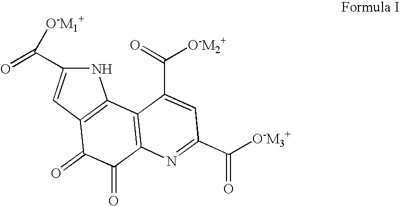
where
M₁ is hydrogen or potassium;
M₂ is hydrogen or potassium;
M₃ is hydrogen or potassium;
provided that at least one of M₁, M₂, and M₃ is not hydrogen.

19. The method of claim 18, provided that at least two of M₁, M₂, and M₃ are not hydrogen.

20. The method of claim 18, provided that M₁, M₂, and M₃ are all potassium.

21. A triacid according to Formula I: 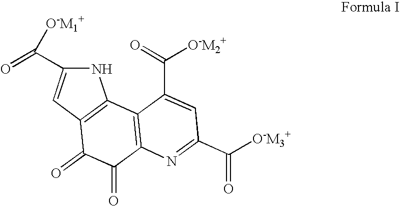
where
M₁ is hydrogen or potassium;
M₂ is hydrogen or potassium;
M₃ is hydrogen or potassium;
provided that at least one of M₁, M₂, and M₃ is not hydrogen.

22. The triacid of claim 21, provided that at least two of M₁, M₂, and M₃ are not hydrogen.

23. The triacid of claim 22, provided that M₁, M₂, and M₃ are all potassium.
